(19) United States
(12) Patent Application Publication
(10) Pub. No.: US 2013/0330419 A1
(43) Pub. Date: Dec. 12, 2013

(54) COMPOSITIONS AND METHODS FOR PERSONAL TUMOR PROFILING TREATMENT

(71) Applicant: Dorit Arad, Tel Aviv (IL)

(72) Inventor: Dorit Arad, Tel Aviv (IL)

(21) Appl. No.: 14/001,023

(22) PCT Filed: Aug. 30, 2012
(86) PCT No.: PCT/US2012/026392

§ 371 Date: Aug. 22, 2013

Related U.S. Application Data
(60) Provisional application No. 61/445,572, filed on Feb. 23, 2011.

(51) Int. Cl. A23L 1/305; G01N 33/50

(52) U.S. Cl. CPC A23L 1/3051; G01N 33/5091
USPC 424/638; 435/34

(57) ABSTRACT

The present invention discloses therapeutic compositions and methods for treating a patient having a tumor disease. Methods, and dietary compositions thereof, for determining a diet regime for a patient with a tumor disease include the steps of: providing a sample of the patient; profiling at least one biochemical parameter of the sample using a biochemical analyzer to obtain a profile; identifying a biologically-active molecular feature of the profile; correlating the feature with a biochemical pathway related to the tumor's metabolism or proliferation; determining the diet regime specific to the patient, wherein the diet regime includes at least one biologically-active molecule corresponding to the feature of the profile; and administering the diet regime to the patient in a therapeutically-effective dosage. Preferably, the sample is selected from the group consisting of: a tumor sample, biological tissue, an organ sample, blood, blood serum, blood plasma, and urine.

### CROSS REFERENCE TO RELATED APPLICATIONS

This patent application claims priority to U.S. Provisional Patent Application No. 61/445,572 filed Feb. 23, 2011, which is hereby incorporated by reference in its entirety.

### FIELD AND BACKGROUND OF THE INVENTION

The present invention relates to therapeutic compositions and methods for treating a patient having a tumor disease. More particularly, the present invention relates to patient-specific dietary compositions and regimes based on laboratory results of personal, individualized body samples and computer modeling which implements analysis and statistics.

It has long been known that conventional medicine alone cannot cure most patients afflicted with cancer. Scientists have been revamping old approaches using the accumulated insight gleaned from research literature and the advent of new research tools. For example, it has been shown that dietary modification can alter the course of tumor formation and development.

In 1965, Lorincz reported in the Nebraska Journal of Medicine a reduced tumor size in cancerous animals treated with a phenylalanine-deprivation diet. In 1969, the benefits of treating advanced cancer patients with a diet restricted in the amino acids phenylalanine and tyrosine were reported (Journal of the American Dietetic Association).

In other reports, it has been shown that animals that were switched from a high-protein to a low-protein diet had significantly reduced tumor growth (35-40%) relative to animals fed with a high-protein diet. Animals that were subsequently switched from a low-protein diet to a high-protein casein diet started growing tumors again. These findings show that nutritional manipulation can turn cancer “on and off.”

Patent application WO 89/06549 discloses depletion of the essential amino acid tryptophan from a body of a tumor patient. Further research has been made which shows correlation between the biochemistry of both normal and cancerous cells and diet, nutrients, phytochemicals, and herbs. For example, the Controlled Amino Acid Therapy (CAAT) protocol of A.P. John creates a deficiency in the amino-acid precursor pool in a patient's body. The CAAT diet is also low in carbohydrates and certain other nutrients in order to sustain a desirable body weight.

Several other studies have reported on the therapeutic benefits of carbohydrate- or glucose-deprivation diets. Kritchevsky, for example, reported that cancerous tumors regress in laboratory animals when their dietary carbohydrates are reduced by 10 percent, and are even eliminated from the body when the carbohydrates are reduced by 40 percent.

The teams of both Lee and Spitz list more than twenty studies supporting their discovery that a glucose-deprivation diet causes apoptosis in cancer cells. Thus, since most cancers depend largely upon carbohydrates or glucose as their major fuel source, the benefits that can be derived by reducing the amount of carbohydrates in the diets of cancer patients are compelling.

It is emphasized that the proposed dietary protocols mentioned above provide a general treatment which is largely based on well-known concepts such as depleting the precursor pools of certain amino acids, or carbohydrate pools, so as to retard tumor growth and angiogenesis (as in the CAAT protocol). Thus, the CAAT method, and other therapies mentioned above, is not individualized to the patient or to a specific tumor.

An increasing number of cancer patients are being treated with complementary therapies as part of their treatment regimen such as CAAT (AP John Institute for Cancer Research). Various scientific journals report on investigations concerning integrative and complementary cancer treatments. Thus, it is now evident that not only conventional drugs, but specific biological compounds, can interfere with specific functions in cancer cells.

However, despite many promising preclinical and clinical studies in recent years, dietary amino-acid restriction and other dietary approaches to cancer treatment have not yet gained wide clinical application. Most clinicians and investigators are probably unfamiliar with nutritional approaches to cancer. Many others may consider amino-acid restriction as an “old idea,” since it has been examined for several decades. New studies have revealed new oncogenes, cancer metabolic pathways, and molecular mechanisms of cancer which enable amino-acid restriction to be re-approached with the aid of modern analytical tools.

It therefore remains a long felt and unmet need to provide novel formulations and methods of treatment which are personalized to individual patients. It would be desirable to provide such therapeutic compositions and methods for, inter alia, treating patients having tumor diseases.

### SUMMARY

It is the purpose of the present invention to provide therapeutic compositions and methods for treating a patient having a tumor disease.

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications, and equivalents that are included in the spirit and scope of the invention, as defined in the appended claims. As used herein and in the claims, the terms and phrases set out below have the meanings which follow. As used herein, the term “biochemical profile” refers in a non-limited manner to a biochemical test or array of tests, usually involving the use of automated instrumentation, performed on individuals or patients. This test or panel of tests is usually selected for its ability in the particular species to evaluate the functional capacity of a tissue, organ, tumor, tissue, blood serum, plasma, or other body sample in the body. The “profile” may literally be the result plotted on individual or parallel numerical scales, producing a pattern such as a plotted graph. In accordance with embodiments of the present invention, the scans or profiles measure proportions of metabolites and biochemicals including: minerals, amino acids, precursors of amino acids, antioxidants, fatty acids, lipids, proteases, protease inhibitors, antagonists, carbohydrates, oligosaccharides, vitamins, nutrients, ions, minerals, trace elements, cofactors or any other metabolite, nutrient, or biological molecule, or a mixture thereof, produced by various metabolic processes, including processes that are associated with cancer metabolism or proliferation.

Non-limiting examples of biochemical profiles or biophysical profiles as herein disclosed may include: a chemosensitivity profile, an amino-acid profile, a genomic profile, a genetic-marker profile, a gene-expression profile, a transcriptome profile, a proteomics profile, a metabolomics profile, a pharmacogenomics profile, a pharmacokinetics profile, an electromagnetic-frequency profile, a electrochemical profile, a fatty-acid profile, a carbohydrate profile, a lipid profile, an oligosaccharide profile, a metabolite profile, a chemical profile, an organic- or inorganic-ion profile, a free-radical profile, a biomarker profile, and an autophagy profile.

Non-limiting examples of a biochemical pathway associated with tumor metabolism or proliferation may include: altered gene expression, gene mutagenesis, metastasis, apoptosis, programmed cell death, autophagy, cell starvation, angiogenesis, growth-factor regulation, receptor regulation, signal transduction, cell proliferation, cell migration, cell adhesion, cell expansion, cell differentiation, cell invasion, tissue-progenitor regulation, cell death, aging process, cellular senescence, carcinogenesis, DNA repair, DNA-damage responses, tumorigenesis, anaplasia, abnormal protein synthesis and expression, genomic instability, neoplasia, thrombosis, hyperplasia, dysplasia, aneuploidy, genomic amplification, variation in nuclear size and shape, abnormal tissue organization, growth signals, immortality, mitosis, cell-cycle regulation, homeostasis, transcription, haploinsufficiency, telomerase mutations, oxidative stress, hypoxia, hyperprolactinemia DNA methylation, pleomorphism, atypia, necrosis, meningitis, astrocytoma, glioblastoma multiforme, and any combination thereof.

Altered gene expression or mutagenesis mentioned above may be associated with genes including among others: oncogenes, tumor-suppressor genes, growth-factor genes, angiogenic-factor genes, receptor genes, and any combination of genes thereof. The term “oncogenes” used herein refers to genes that produce growth factors and other substances that signal to a cell to grow, differentiate, and divide into daughter cells. Tumor-suppressor genes, such as the p16, p53, and BRCA1 genes, normally produce a negative growth factor that stops the division of cells. The abnormally-inactivated tumor-suppressor gene and/ or the abnormally-activated oncogene are inherited by each of the daughter cells, causing tumorous tissue to develop.

The present invention relate the generalized concepts of dietary amino-acid restriction, and the metabolic difference of cancer cells that causes the cells to consume some nutritional components (e.g. glucose, fat, or glutamine) in excess. Such approaches, either alone or in combination with other treatments, are utilized in developing personal-diagnostic protocols according to specific tumor type and molecular-diagnostic results. Such protocols, inter alfa, enable the determination of personally-prescribed formulations that can be administered and adjusted during a treatment cycle.

The personalized, therapeutic, biological protocols described herein provide significant benefits. The protocols include administering biological molecules which are specifically formulated according to input data associated with a patient's tumor. Thus, such therapies are more effective in preventing the specific tumor's development in a specific patient. In addition, the biological compositions and therapies of the present invention allow oncologists the option to choose for the patient a chemotherapy regimen that is more effective when administered with such biological compositions, enabling the reduction of the large amounts of drugs or radiation therapy (which are often toxic) required to inhibit tumor growth. Furthermore, functional-food therapies described in the present invention can be self-administered at one's convenience. Such treatments are more patient-friendly, increasing compliance and improving the patient's quality of life.

It is noted that the term “exemplary” is used herein to refer to examples of embodiments and/or implementations, and is not meant to necessarily convey a more-desirable use-case. Similarly, the term “preferred” is used herein to refer to an example out of an assortment of contemplated embodiments and/or implementations, and is not meant to necessarily convey a more-desirable use-case. Therefore, it is understood from the above that “exemplary” and “preferred” may be applied herein to multiple embodiments and/or implementations.

Therefore, according to the present invention, there is provided for the first time a method for determining a diet regime for a patient with a tumor disease, the method including the steps of: (a) providing a sample of the patient; (b) profiling at least one biochemical parameter of the sample using a biochemical analyzer to obtain a profile; (c) identifying a biologically-active molecular feature of the profile; (d) correlating the feature with a biochemical pathway related to the tumor's metabolism or proliferation; (e) determining the diet regime specific to the patient, wherein the diet regime includes at least one biologically-active molecule corresponding to the feature of the profile; and (f) administering the diet regime to the patient in a therapeutically-effective dosage.

Preferably, the sample is selected from the group consisting of: a tumor sample, biological tissue, an organ sample, blood, blood serum, blood plasma, and urine.

According to the present invention, there is provided for the first time a method for diagnosing a tumor in a subject, the method including the steps of: (a) providing a sample of the subject; (b) profiling at least one biochemical parameter or at least one biophysical parameter of the sample using a biochemical analyzer or a biophysical analyzer to obtain a profile; (c) storing results of the profile in a profile database; and (d) processing the results by comparing the results with profile data in the database, thereby diagnosing the tumor.

Preferably, the sample is selected from the group consisting of: a tumor sample, biological tissue, an organ sample, blood, blood serum, blood plasma, and urine.

Preferably, the step of processing includes calculating the ratio between a difference in a profiled, biological-molecule value of a healthy individual and a tumor patient, and a healthy biological-molecule value of a healthy individual.

Preferably, the step of processing includes comparing biochemical profile data of the sample with corresponding profile data of healthy tissue of the subject.

Preferably, the profile is selected from the group consisting of: a chemosensitivity profile, an amino-acid profile, a genomic profile, a deprivation-state profile, a genetic-marker profile, a gene-expression profile, a transcriptome profile, a proteomics profile, a metabolomics profile, a pharmacogenomics profile, pharmacokinetics profile, an electromagnetic-frequency profile, an electrochemical profile, a fatty-acid profile, a carbohydrate profile, a lipid profile, an oligosaccharide profile, a metabolite profile, a chemical profile, an organic-ion profile, or an inorganic-ion profile, a free-radical profile, a bioimpedance profile, a conductivity profile, a voice-analysis profile, a biomarker profile, and any combination thereof.

According to the present invention, there is provided for the first time a dietary composition for a patient with a tumor, the composition including depleted or reduced amino-acid concentrations of at least 50% reduction from normal consumption to depletion of at least one amino acid selected from the group consisting of: Arginine (Arg), Glutamine (Gin), Methionine (Met), Asparagine (Asn), Phenylalanine (Phe), Histidine (His), Glycine (G1t), Tryptophan (Trp), Leucine (Leu), Threonine (Thr), Valine (Val), Cystine (Cys), Isoleucine (Iso), Lysine (Lys), Aspartic acid (Asp), and Tyrosine (Tyr).

Preferably, the tumor is associated with breast cancer, and wherein the dietary composition includes depleted or reduced amino-acid concentrations of at least 50% reduction from normal consumption to depletion of at least one of: Arg, Gln, Met, Asn, Phe, and His.

Preferably, the tumor is associated with prostate cancer, and wherein the dietary composition includes depleted or reduced amino-acid concentrations of at least 50% reduction from normal consumption to depletion of at least one of: Gln, Gly, Trp, Arg, Leu, His, and Met.

Preferably, the tumor is associated with lung cancer, and wherein the dietary composition includes depleted or reduced amino-acid concentrations of at least 50% reduction from normal consumption to depletion of at least one of: His, Gln, Asn, Cys, Leu, Met, and Trp.

Preferably, the tumor is associated with colorectal cancer, and wherein the dietary composition includes depleted or reduced amino-acid concentrations of at least 50% reduction from normal consumption to depletion of at least one of: Thr, Gly, Met, Cys, Phe, Tyr, Trp, Asn, and Val.

Preferably, the tumor is associated with head and neck cancer, and wherein the dietary composition includes depleted or reduced amino-acid concentrations of at least 50% reduction from normal consumption to depletion of at least one of: Met, Cys, Tyr, Leu, and Asp.

Preferably, at least one molecule is selected from the group consisting of: a precursor of at least one feature, an antagonist of at least one feature, an inhibitor of at least one feature, a participant in a biochemical pathway associated with at least one feature, a cofactor of the feature, a participant in a biochemical pathway associated with the metabolism or the proliferation, and a biomarker of a tumor disease.

Preferably, the biochemical pathway is selected from the group consisting of: of altered gene expression, gene mutagenesis, metastasis, apoptosis, programmed cell death, autophagy angiogenesis, growth factor regulation, receptor regulation, signal transduction, cell proliferation, cell migration, cell adhesion, cell expansion, cell differentiation, cell invasion, tissue progenitors regulation, cell death, aging process, cellular senescence, carcinogenesis, DNA repair, DNA-damage responses, tumorigenesis, anaplasia, abnormal protein synthesis and expression, genomic instability, neoplasia, thrombosis, hyperplasia, dysplasia, aneuploidy, genomic amplification, variation in nuclear size and shape, abnormal tissue organization, growth signals, immortality, mitosis, cell cycle regulation, homeostasis, transcription, haploinsufficiency, telomerase mutations, telomerase mutations, oxidative stress, hypoxia, hyperprolactinemia DNA methylation, pleomorphism, atypia, necrosis, meningitis, astrocytoma, glioblastoma multiforme, deprivation state target, autophagy and any combination thereof.

According to the present invention, there is provided for the first time a dietary composition for a patient with a tumor, the composition including: at least one biologically-active molecule corresponding to at least one biologically-active molecular feature of a biochemical tumor profile, wherein at least one feature correlates with at least one biochemical pathway related to metabolism or proliferation of the tumor.

Preferably, at least one feature is either reduced, lacking, or in excess in the tumor profile relative to a healthy profile of corresponding healthy tissue.

Preferably, the identity and/or dosage of at least one molecule is either in direct correlation or in inverse correlation with at least one feature or in correlation modified by a constant thereof.

Preferably, at least one molecule is selected from the group consisting of: a precursor of at least one feature, an antagonist of at least one feature, an inhibitor of at least one feature, a participant in a biochemical pathway associated with at least one feature, a cofactor of the feature, a participant in a biochemical pathway associated with the metabolism or the proliferation, and a biomarker of a tumor disease.

Preferably, the molecule is adapted to cause cells in the tumor to starve, resulting in signals and/or processes to be triggered, and wherein the signals and/or processes include AKT, HSP, HSP70, autophagy, and apoptosis.

Preferably, the biochemical pathway is selected from the group consisting of: of altered gene expression, gene mutagenesis, metastasis, apoptosis, programmed cell death, autophagy angiogenesis, growth factor regulation, receptor regulation, signal transduction, cell proliferation, cell migration, cell adhesion, cell expansion, cell differentiation, cell invasion, tissue progenitors regulation, cell death, aging process, cellular senescence, carcinogenesis, DNA repair, DNA-damage responses, tumorigenesis, anaplasia, abnormal protein synthesis and expression, genomic instability, neoplasia, thrombosis, hyperplasia, dysplasia, aneuploidy, genomic amplification, variation in nuclear size and shape, abnormal tissue organization, growth signals, immortality, mitosis, cell cycle regulation, homeostasis, transcription, haploinsufficiency, telomerase mutations, telomerase mutations, oxidative stress, hypoxia, hyperprolactinemia DNA methylation, pleomorphism, atypia, necrosis, meningitis, astrocytoma, glioblastoma multiforme, deprivation state target, autophagy and any combination thereof.

Most preferably, the altered gene expression or mutagenesis is associated with genes selected from the group consisting of: oncogenes, tumor suppressor genes, growth factor genes, angiogenic factor genes, receptor genes, and any combination thereof.

Preferably, at least one molecule is selected from the group consisting of: amino acids, precursors of amino acids, antioxidants, fatty acids, lipids, proteases, protease inhibitors, antagonists, carbohydrates, oligosaccharides, vitamins, nutrients, ions, minerals, trace elements, cofactors, enzymes, enzyme inhibitors, and a mixture thereof.

Preferably, the composition further including: a predetermined amino-acid content correlated with the amino-acid profile of the tumor.

Most preferably, the composition further including: at least one composition attribute selected from the group consisting of: complex carbohydrates, 0% fat, 0% glucose, 0% fructose, and 0% carbohydrates, arctigenin, at least one depleted amino acid, at least one depleted amino-acid precursor, at least one depleted metabolite involved in synthesis of the amino acid.

Preferably, the composition further including: at least one depleted, essential amino acid selected from the group consisting of: Isoleucine, Leucine, Lysine, Methionine, Phenylalanine, Threonine, Tryptophan and Valine, a precursor thereof, a metabolite involved in synthesis of the amino acid, and enzymes capable of decomposing the amino acid.

Preferably, the composition further including: at least one depleted, non-essential amino acid selected from the consisting of: Alanine, Asparagine, Aspartic acid, Cysteine, Glutamic Acid, Glutamine, Glycine, Proline, Selenocysteine, Serine, Tyrosine, Arginine, Histidine, Ornithine, and Taurine, a precursor thereof, and a metabolite involved in synthesis of the amino acid.

Preferably, the tumor profile is associated with a tumor selected from the group consisting of: a sarcoma, carcinoma, lymphoma, myeloma leukemia, and cancers of the central nervous system (CNS).

Preferably, the composition adapted to provide a synergistic therapeutic effect with respect to inhibition of tumor metabolism and/or proliferation when administered in combination with a conventional anti-tumor drug or treatment to the patient.

According to the present invention, there is provided for the first time a method for optimizing and administering a diet treatment for a patient with a tumor disease, the method including the steps of: (a) administering a dietary composition to the patient in a therapeutically-effective dosage; (b) profiling amino acids of a tumor of the patient using a biochemical analyzer; (c) monitoring the tumor for indication of a deprivation state using a bioanalytical tool; (d) detecting the deprivation state of the tumor; and (e) second-stage administering of a modified deprivation diet in a therapeutically-effective dosage, wherein the modified deprivation diet includes the dietary composition, at least one participant in a biochemical pathway associated with metabolism or proliferation of the tumor, and at least one cytotoxic material.

According to the present invention, there is provided for the first time a system for processing tumor-related profile data, the system including: (a) a CPU for performing computational operations; (b) a memory module for storing data; (c) a profile-analysis module for profiling at least one biochemical parameter or at least one biophysical parameter of a tumor sample; (d) a profile-processing module for processing profiling results of at least one biochemical parameter or at least one biophysical parameter; and (e) a protocol-generating module for generating a protocol, for a dietary composition based on the processing of the profiling results.

According to the present invention, there is provided for the first time a non-transitory computer-readable medium, having computer-readable code embodied on the non-transitory computer-readable medium, the computer-readable code including:

(a) program code for receiving a profile of at least one biochemical parameter or at least one biophysical parameter of a tumor sample of a subject using a biochemical analyzer or a biophysical analyzer; (b) program code for storing results of the profile in a profile database; and (c) program code for processing the results by comparing the results with profile data in the database, thereby diagnosing the sample.

These and further embodiments will be apparent from the detailed description and examples that follow.

### BRIEF DESCRIPTION OF THE DRAWINGS

The present invention is herein described, by way of example only, with reference to the accompanying drawings, wherein:

FIG. 1 is a simplified flowchart of the major operational steps for determining a diet regime for a patient with a tumor disease, according to preferred embodiments of the present invention;

FIG. 2 is a simplified flowchart of the major operational steps for diagnosing a tumor in a subject, according to preferred embodiments of the present invention;

FIG. 3 is a simplified flowchart of the major operational steps for an exemplary profile processing system, according to preferred embodiments of the present invention;

FIG. 4A is a plotted-graph depiction of an exemplary amino-acid profile of a tumor sample, according to preferred embodiments of the present invention;

FIG. 4B is a plotted-graph depiction of an exemplary computer-generated profile for a dietary composition formulated according to the amino-acid profile of FIG. 4A, according to preferred embodiments of the present invention;

FIG. 5 is a high-level schematic block diagram of a total personal profiling food system, showing processing steps, for creating personal combinations for patients according to their cancer type and profile, according to preferred embodiments of the present invention;

FIG. 6 is a simplified flowchart of the major operational steps for optimizing and monitoring the treatment, changing the formulation with progression of treatment, and formulating and administering the diet, according to preferred embodiments of the present invention;

FIG. 7 is a simplified flowchart of the major operational steps for a drug-supplement cancer-treatment plan using an AA starvation diet and drug supplements in-vitro to derive an optimal chemotherapy, drug supplement, diet plan, according to preferred embodiments of the present invention.

### DESCRIPTION OF THE PREFERRED EMBODIMENTS

The present invention relates to therapeutic compositions and methods for treating a patient having a tumor disease. The principles and operation for such compositions and methods, according to the present invention, may be better understood with reference to the accompanying description and drawings.

Preferred embodiments of the present invention include biologically-active compositions and/or protocols that are personalized to a specific patient and/or to a specific tumor type. Such patient-specific biological compositions and therapies can be administered to interfere with tumor development. Such biological compositions can be administered in-vitro along with conventional treatment protocols to provide an optimal combination for synergistically managing cancers successfully.

Preferred embodiments of the present invention provide a personalized therapy or treatment, preferably for tumor patients, which serve to link the personalized biochemical data obtained by various diagnostic tools and the personalized medical treatment associate with dietary formulation.

In some embodiments of the present invention, a specific profile is made of an individual tumor sample or any body sample from the patient, providing “fingerprint” profile characteristics of the tumor. The differences between the tumor fingerprint profile and the profile of other healthy organs of the individual (or a standard profile for a healthy individual) are used as input data for processing and modeling. Such profile data is used to tailor a specific diet or composition to the individual patient in order to interfere with tumor development. Such a treatment would be more efficacious in preventing tumor growth in the specific patient than other population-generalized, conventional cancer treatments.

In other embodiments of the present invention, a dietary solution is provided based on molecular profiles for populations at risk for cancer. There is a great need to prevent cancer in populations with inherited risk (e.g. the Brca2 gene). Personal diet prescription based on the specific metabolic and structural body features are suggested to lower the risk of acquiring cancer for such populations. In some embodiments, a protocol is provided for defining a personalized diet based on amino acid (AA) and metabolic deprivation according to the molecular profile of a person as determined using bioinformatics.

Other embodiments of the present invention provide a method for using personalized, patient data for constructing a personalized, dietary formula for a general tumor type according to results of plasma, blood, serum, urine, or tissue profiles of a specific person's tumor compared to the mean profile associated with healthy individuals. Such dietary formula can also be constructed according to deviations from a general AA profile for the cancer type, as well as according to a specific person's specifications (e.g. a blood plasma characteristics, tissue structure, and metabolic nature of the tumor).

Other embodiments of the present invention provide methods for starving a tumor using a personalized, dietary medicament, and monitoring the starvation stage via biochemical tests known in the art. In other embodiments, terahertz imaging is used to determine the stage of starvation, or to detect a biomarker that is typical of a starvation stage.

Once a starvation stage is reached, the treatment enters its second stage, providing deprivation-state inhibitors for preventing the tumor from seeking food in the surrounding vicinity which results in cell death. An example for such an inhibitor is arctigenin, which is an AKT1 and HSP70 inhibitor. Such compounds are included in the food regimen in this second stage.

Other embodiments of the present invention provide a whole system for optimizing the chemotherapy regimen that a cancer patient receives by: creating a personalized, starvation-diet prescription as described above (using literature data, pathological data of tumor type, personal AA structure, and metabolic profiling data from the patient's body sample), creating a personalized, drug-supplement formula (using literature data, pathological data of tumor type, and molecular-diagnostic tests which indicate the participation of specific proteins such as COX-2 or aromatase in the proliferation process), performing an in-vitro chemosensitivity test (using methods known in the art), pre-treating the tumor tissue with the dietary and drug-supplement formula, treating the tumor tissue with drug protocols and drug-protocol combinations to access the optimal sensitivity for the specific tumor, and providing the results to the physician to administer a treatment package containing an optimal food regimen along with optimal drug supplements and optimal chemotherapy drugs.

In addition, a metabolic difference of cancer cells is known due to the Warburg effect, causing the cells to consume some nutritional components such as glucose, fats, or glutamine in excess. Restriction of these nutrients in a cancer patient's diet according to the exact metabolic type, in an overall nutrition plan, using prepared functional-food combinations is an embodiment of the present invention.

Thus, combinations in the functional-food regime are prescribed according to the following criteria: type of cancer from pathology, AA profile based on cancer type, stage of cancer based on personal AA profile, metabolic type of tumor based on cancer type and metabolic test, and tolerance of the tumor cell to starvation.

Preferred embodiments of the present invention provide a dietary composition for a patient with a tumor. Such dietary compositions include at least one biologically-active molecule corresponding to a biologically-active molecular feature (or a precursor, antagonist, inhibitor, or cofactor of a biologically-active molecular feature) of a biochemical profile of the patient's tumor. The biologically-active molecular feature correlates with a biochemical pathway related to tumor metabolism or proliferation. The biologically-active molecular feature is either reduced, depleted, or in excess in the patient's tumor profile relative to a profile of a corresponding healthy standard.

In other embodiments of the present invention, a dietary composition is provided for a patient with a tumor. The dietary composition is derived from, and linked to, one or more diagnostic tests used to reveal the metabolic nutrient consumption specific to that tumor.

In preferred embodiments of the present invention, the biologically-active molecule may include: amino acids, precursors of amino acids, antioxidants, fatty acids, lipids, proteases, protease inhibitors, antagonists, carbohydrates, oligosaccharides, vitamins, nutrients, ions, minerals, trace elements, cofactors or any other metabolite, and a mixture thereof.

In preferred embodiments of the present invention, the identity and amount of the biologically-active molecule within the dietary composition is either directly or inversely correlated with the biological molecular feature of the patient's tumor profile modified by a constant. In some embodiments, the biochemical profile used in such a correlation is processed via a correlation algorithm to provide a patient-specific, treatment profile. Such a treatment profile can be used to formulate a dietary composition and regime thereof specific to the tumor patient.

Technologies have recently been developed to analyze amino acids with high accuracy. An AA profile can be performed with any conventional AA analyzer or separating apparatus (e.g. automated chromatographic instruments designed for analytical methodologies such as a low-pressure or High-Pressure Liquid Chromatograph (HPLC) capable of generating mobile-phase gradients that separate AA analytes on a chromatographic column, and are detected on a mass spectrometer (MS or LC-MS)) known in the art (see Shimbo K., et al. (2009) Biomed Chromatogr. 24: 683-691).

In preferred embodiments of the present invention, the dietary composition includes a predetermined AA content correlated with the AA profile of the patient's tumor or plasma concentrations. In such embodiments, the dietary composition is depleted of at least one amino acid, an AA precursor thereof, or a metabolite involved in the synthesis of the amino acid. In such embodiments, an amino acid (or other biological molecular) deprivation therapy is used in treating cancer patients.

Such an amino acid or biological deprivation diet is intended to impair the development of cancer cells by interfering with tumor metabolism and biochemical proliferation processes. Such a biological approach, involving the deprivation of metabolites and other active biological molecules such as carbohydrates and certain amino acids, can inhibit DNA and protein synthesis, angiogenesis, and also curtail mitotic signal transduction receptors on cellular membranes of cancer cells. The latter process may affect receptor regions common to numerous, tumor growth factors.

Thus, the compositions and diet regimes described herein not only enhance conventional medicine, they also allow treatment of cancer patients using less toxic therapies, but enhance chemotherapy as well, and can be chosen according to lab tests. Such a daily diet may include specific amounts of: certain metabolites, biological molecules such as amino acids, carbohydrates, fatty acids, vitamins, minerals and combinations thereof formulated according to a biochemical profile of the patient's tumor. Such compositions and treatment methods deplete or reduce the level of a biologically-active molecule in the body which is found to be in excess in a patient's tumorous tissue profile relative to a corresponding healthy tissue profile.

It is well known that amino acids are the building blocks of all proteins. Twenty-two amino acids are commonly classified as essential to life. Fourteen of these can be synthesized within the body, and are classified as non-essential, whereas the remaining eight, classified as essential, must be provided by the daily diet.

In further embodiments of the present invention, the dietary composition is depleted of or is characterized by a reduced level of an essential amino acid such as: Isoleucine, Leucine, Lysine, Methionine, Phenylalanine, Threonine, Tryptophan, Valine, a precursor thereof, or a metabolite involved in the synthesis of the amino acid. In other embodiments, the dietary composition is depleted of or is characterized by a reduced level of a non-essential amino acid such as: Alanine, Asparagine, Aspartic acid, Cysteine, Glutamic Acid, Glutamine, Glycine, Proline, Selenocysteine, Serine, Tyrosine, Arginine, Histidine, Ornithine, Taurine, a precursor thereof, or a metabolite involved in the synthesis of the amino acid.

It is herein acknowledged that there are four amino acids essential to synthesis of DNA. These include glycine, glutamic acid, aspartic acid, and serine. In some embodiments of the present invention, the dietary composition is adapted to decrease the level of specific amino acids within the body (e.g. amino acids essential to DNA synthesis), thereby inhibiting the cancer cell's development and proliferation. Such dietary compositions affect tumor growth (e.g. by inhibiting DNA synthesis) when administered alone or synergistically with conventional chemotherapeutic drugs known to inhibit DNA synthesis in cancer cells.

Embodiments of the present invention provide methods for reducing the precursor pool of certain amino acids, especially amino acids highly expressed in a specific tumor profile, resulting in the cancer cell's ability to produce sufficient proteins for self-replication being disrupted.

Embodiments of the present invention further enable the identification and profiling of specific, established, predetermined biomarkers of specific tumor types and specific stages of tumor development. The biomarkers may include prognostic markers and/or predictive markers for specific therapies. Such a profiling of tumor biomarkers enables the selection of a specific therapy for a specific tumor patient, and further enables tumor response to a certain therapy to be predicted.

Such biomarker profiling and analysis is based on different platforms including protein expression by immunohistochemistry (IHC), messenger RNA (mRNA) level by polymerase chain reaction (PCR), gene-copy number by fluorescence in-situ hybridization (FISH), or comparative, genomic hybridization and proteomic profiling. Embodiments of the present invention use different quantitative platforms of profiling a tumor sample or body sample, especially those encompassing biochemical and biophysical analysis, in order to produce computer-generated, adapted therapy outcomes that cannot be achieved by standard methods.

Since cancer cells depend upon angiogenesis for their growth and reproduction much more than normal cells, compared with normal cells. Depriving cancer cells of amino acids essential for their growth impacts the building of new blood vessels. An example of such a protein, essential to the establishment of new blood vessels, is elastin. Elastin contains five amino acids in its protein sequence: glycine, proline, leucine, isoleucine, and valine. In other embodiments of the present invention, treatment methods and dietary compositions are provided which target cancer cells due to such cells being significantly more sensitive to deletion of these five amino acids.

Reference is now made to the role of carbohydrates in the diets of cancer patients. Most cancers depend largely upon carbohydrates or glucose as their major fuel source. It has been recently published (Medical Hypotheses) that cancer cells cannot use carbohydrates or fats in their mitochondria, as do normal cells, but must rely almost exclusively upon glycolysis and the metabolism of glucose for their daily energy. It has been further shown (Lee and others) that cancer cells enter apoptosis when deprived of glucose. Other metabolic pathways such are beta-oxidation (in which the cancerous cells consume fat) and glutamine dependency are known to exist.

In accordance with embodiments of the present invention, the carbohydrate profile of a tumor sample is assessed and used as the basis for a dietary formulation that is adapted to interfere with individual tumor proliferation.

Additional biochemical pathways or targets that the biologically-active molecule of the dietary composition may be associated with include receptor regions common to numerous, tumor growth factors. It is herein acknowledged that cancer cells use such receptor regions to transmit mitogenic signals into their nuclei. Non-limiting examples of tumor growth factors include: Tyrosine Kinase (TK), Ras Protein (RP), Epithelial Growth Factor (EGF), hepatocellular growth factor, vascular endothelial growth factor, and Insulin-like Growth Factor-1 (IGF-1). Affecting the AA content of a tumorous tissue by administering daily to the patient a dietary composition depleted of certain amino acids may interfere with specific, tumor growth-factor expression and activity, thereby impairing tumor development.

The COX-2 enzyme is an example of a mitogenic factor found to be over-expressed in most cancers. A natural COX-2 inhibitor, such as tumeric, may be optionally included in a dietary composition of the present invention.

It is acknowledged herein that almost every cancerous tumor requires tumor factors for growth and metastasis; normal cells do not have the same necessity. With the exception of steroid hormones, almost all tumor growth factors (e.g. EGF, hepatocellular growth factor, IGF-1, vascular endothelial growth factor, and the Ras gene protein growth factor) are proteins composed of amino acids. Thus, profiling the AA pattern of a specific tumorous tissue enables the formulation of a dietary composition which corresponds to the AA content of the specific tumorous tissue. Such a composition interferes effectively and specifically with tumor metabolism and proliferation.

In other embodiments of the present invention, a daily diet is provided for tumor patients. Such dietary compositions are preferably reduced or depleted in certain active biochemical molecules, metabolites, or nutrients (e.g. specific amino acids, carbohydrates, or fats) according to the biochemical tests performed on the body samples.

Embodiments of the present invention further provide a therapeutic biological protocol individualized to a specific tumor or patient for interfering with specific functions in cancer cells, thereby inhibiting cancer development. In other embodiments, the deprivation of the cells of phosphorus, which is an essential constituent of ATP, GTP, UTP, and CTP (all of which control the function of every metabolic reaction that occurs in every cell of the human body), is employed to impair the metabolism of the cancer cells. A low phosphorus diet would create a deficiency of ATP. Consequently, glycolysis, for example, is impaired, impairing cancer cells ability to grow and reproduce.

Cancer cells differ from normal cells in their ability to survive starvation processes. Normal cells can go into a “dormant” mode and later revive; cancer cells, if starved, undergo apoptosis. Therefore, cancer cells have mechanisms to overcome starvation. These mechanisms are: autophagy (in which the cell starts to digest itself, obtaining its food internally), HSP (heat shock proteins which release signals to the neighboring environment to provide the necessary nutrients), and AKT (kinases which produce the signals mentioned above). All these processes occur when the cancer cells begins to feel starvation. The existence of the factors mentioned above can be detected.

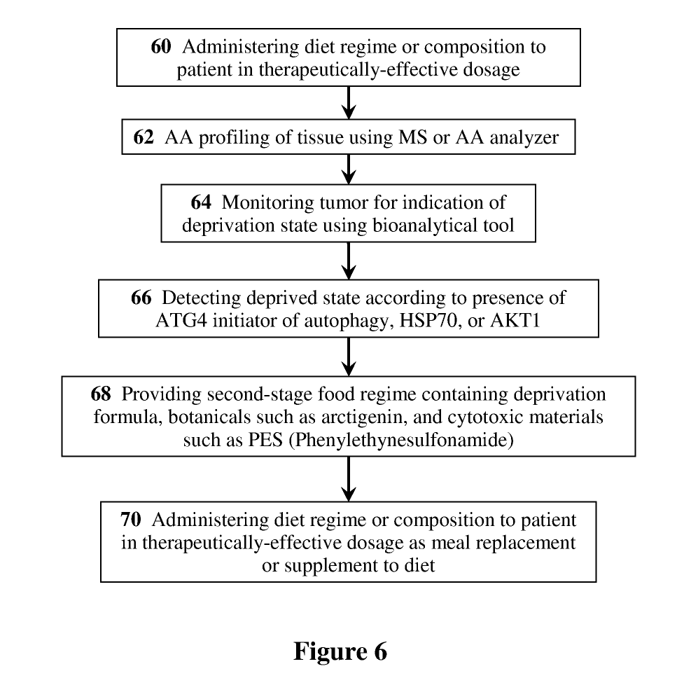

In such a starvation state, cancer cells either undergo autophagy, or become much more aggressive than normal cells. Such starved cancer cells produce HSP70 and AKT1 which decompose neighboring tissue via catabolism. In such a situation, a test can be performed to diagnose the deprivation “state” of the patient's tumor in order to provide a second-generation formula for administering (as described below in Example 9 with regard to FIG. 6).

Embodiments of the present invention provide diet protocols aimed at bringing the cancer cells quickly to a starvation state that can be measured. Once starvation of the cancerous cells has been initiated, the patient receives the deprivation diet, arctigenin, or any other inhibitor of one of the enzymes that participate in the process (e.g. autophagy inhibitors, HSP79, HSP90 inhibitors, and other AKT inhibitors). Arctigenin is used as an example because it is a natural compound.

The diet regime combined with such starvation-factor inhibitors, particularly arctigenin, can be used as an optimized treatment. It is further possible to include arctigenin in the deprivation diet at the initial stage (without requiring monitoring). Such a diet regimen allows the cancer cells starve, without the cancer cells consuming themselves or neighboring cells, resulting in the cells having a reduced tolerance to the starvation and dying. It was recently shown that a leucine-free diet causes an interference with the autophagy mechanism without any inhibitor added. The addition of inhibitors would enable the attack of more cancer mechanisms.

In some embodiments of the present invention, protocols include monitoring a patient's blood chemistry and AA profile regularly (e.g. monthly). In such embodiments, the biochemical profiles are obtained by measuring a variety of biochemical parameters in the blood.

The dietary compositions of the present invention may be taken for approximately six to nine months in three-week treatment intervals followed intermittently by a one-week “pause” in dietary treatment. During the intervening week (i.e. the week-long pause), the patient returns to an otherwise-prescribed, more-balanced diet with proper nutritional supplements.

In some embodiments of present invention, compositions and methods can be applicable to a variety of biochemical profiling involving genotype profiles, molecular markers, DNA sequencing, SNP profiling, microsatellite analysis, and short tandem repeats (STR) analysis.

MS instruments mainly consist of three module: an ion source for converting gas-phase sample molecules into ions (or, in the case of electrospray ionization, for transferring ions that exist in solution into the gas phase), a mass analyzer for sorting the ions by their masses by applying electromagnetic fields, a detector for measuring the quantity of mass-to-charge ratio value, providing data for calculating the abundances of each ion present.

Such MS techniques have both qualitative and quantitative uses including identifying unknown compounds, determining the isotopic composition of elements in a molecule, determining the structure of a compound by observing its fragmentation, quantifying the amount of a compound in a sample, and studying the fundamentals of gas-phase ion chemistry (i.e. the chemistry of ions and neutrals in a vacuum). It is further acknowledged herein that MS is now in very common use in analytical laboratories that study physical, chemical, or biological properties of a variety of compounds.

Reference is now made to MALDI-TOF, a combination of a matrix-assisted laser desorption/ionization source with a time-of-flight mass analyzer. A common example for such a combination is gas chromatography-mass spectrometry (GC-MS). In this technique, a gas chromatograph is used to separate different compounds. It is further acknowledged herein that MS produces various types of data. The most common data representation is the mass spectrum. Certain types of MS data are best represented as a mass chromatogram. The types of chromatograms include selected ion monitoring (SIM), total ion current (TIC), and selected reaction monitoring (SRM).

In preferred embodiments of present invention, the compositions and diet regimes described herein are directed to treat, in a non-limiting manner, tumors including sarcoma, carcinoma, lymphoma, myeloma leukemia, as well as cancers of the Central Nervous System (CNS) and various cancer types such as lung, prostate, and gastric cancer.

In further embodiments of present invention, dietary compositions are formulated using a processing system and/or algorithm configured to determine the identity and/or dosage of the biologically-active molecule according to input of a biochemical profile of the patient tumor.

In the above description, it is to be understood that the mention of preferred embodiments is exemplary only. The present invention will now be further elucidated by the following Examples. The following Examples are presented in order to more fully describe certain embodiments of the present invention. They are in no way, however, meant to be construed as limiting the broad scope of the present invention. A person having ordinary skill in the art can readily devise many variations and modifications of the principles disclosed herein without departing from the spirit and scope of the present invention.

The database referred to herein may contain biochemical and biophysical data of at least the following types: data on different classifications of tumor types, data on normal tissues, data on individuals, and data on populations.

### EXAMPLES

### Example 1

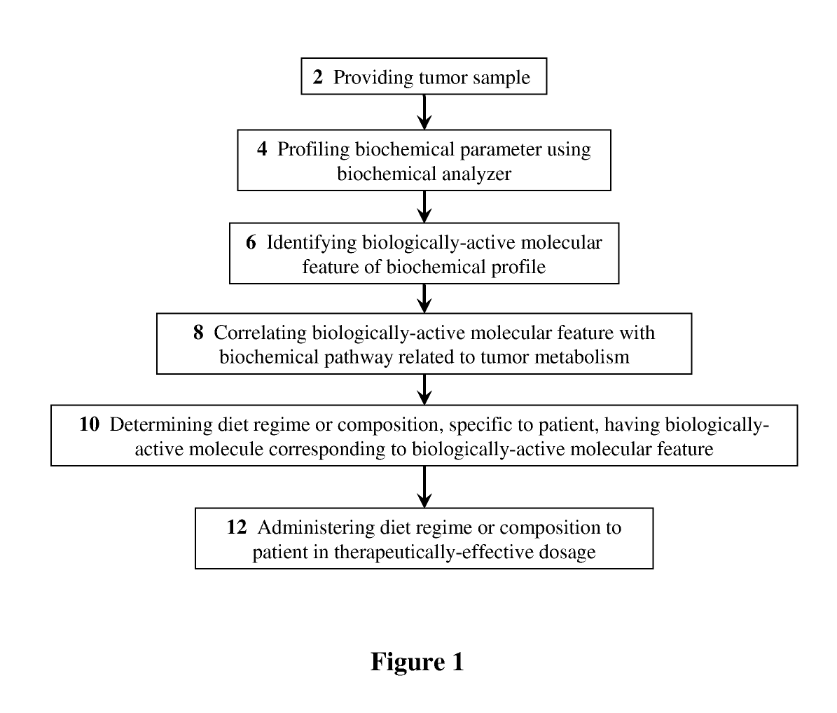

Referring now to the drawing, FIG. 1 is a simplified flowchart of the major operational steps for determining a diet regime for a patient with a tumor disease, according to preferred embodiments of the present invention. The method starts with providing a tumor sample of a patient (Step 2). One or more biochemical parameter of the tumor sample is then profiled using a biochemical analyzer (Step 4). A biologically-active molecular feature of the biochemical profile is then identified (Step 6), and correlated with a biochemical pathway related to the tumor's metabolism or proliferation (Step 8). A diet regime specific to the tumor patient is then determined (Step 10). The diet regime includes administration of a biologically-active molecule corresponding to the biologically-active molecular feature of the biochemical profile. The diet regime is then administered to the patient in a therapeutically-effective dosage (Step 12).

### Example 2

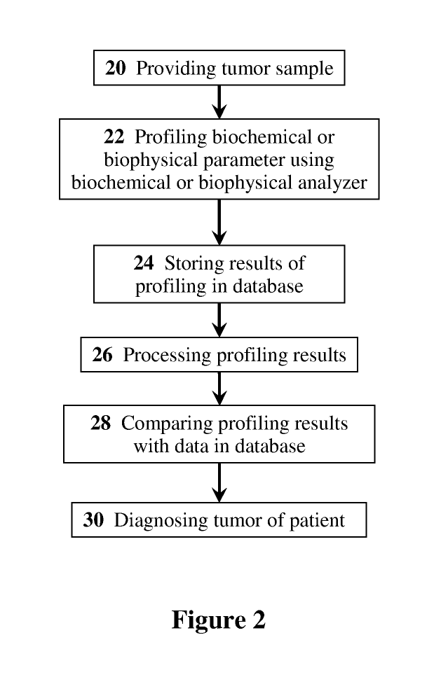

FIG. 2 is a simplified flowchart of the major operational steps for diagnosing a tumor in a subject, according to preferred embodiments of the present invention. The method starts with providing a tumor sample of a subject (Step 20). One or more biochemical or biophysical parameter of the tumor sample is then profiled using a biochemical or biophysical analyzer (Step 22). The results of the profiling are then stored in a database (Step 24), and processed (Step 26). The profiling results are then compared with the data in the database (Step 28), thereby diagnosing the tumor of the subject (Step 30).

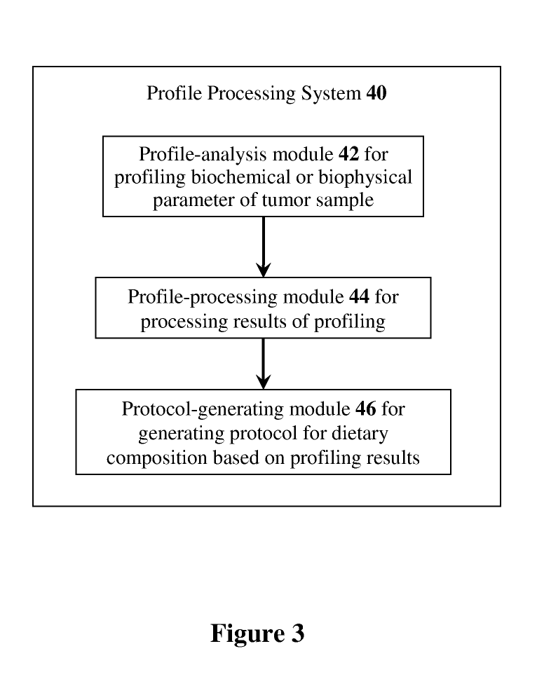

FIG. 3 is a simplified flowchart of the major operational steps for an exemplary profile processing system, according to preferred embodiments of the present invention. Such a profile processing system 40 includes a profile-analysis module 42 for profiling a biochemical or biophysical parameter of a tumor sample, a profile-processing module 44 for processing the results of the profiling, and a protocol-generating module 46 for generating a protocol for a dietary composition based on the profiling results.

### Example 3

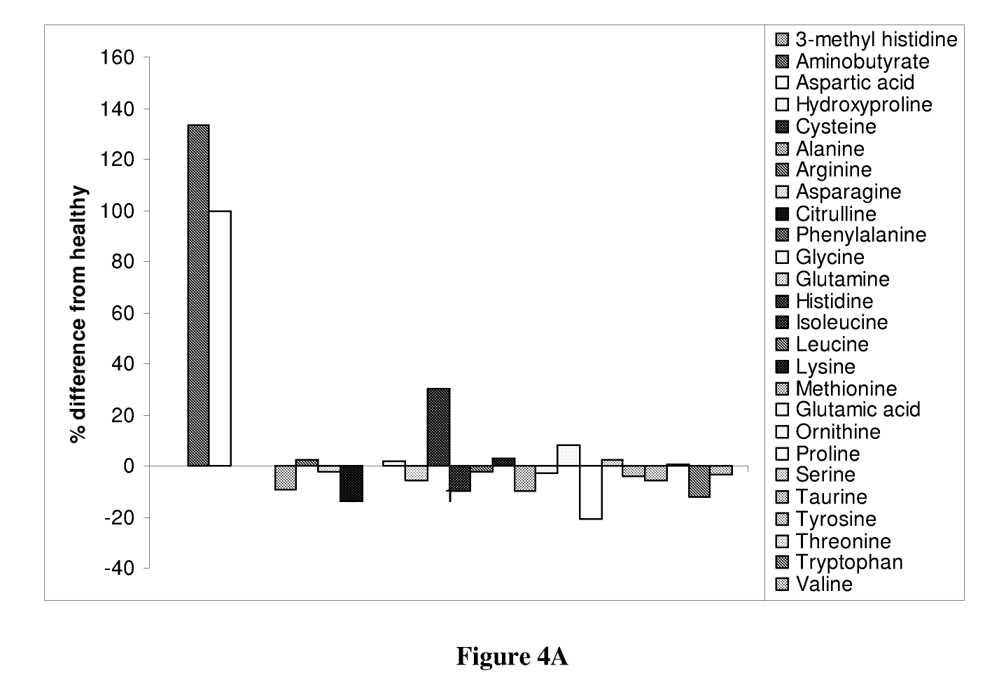

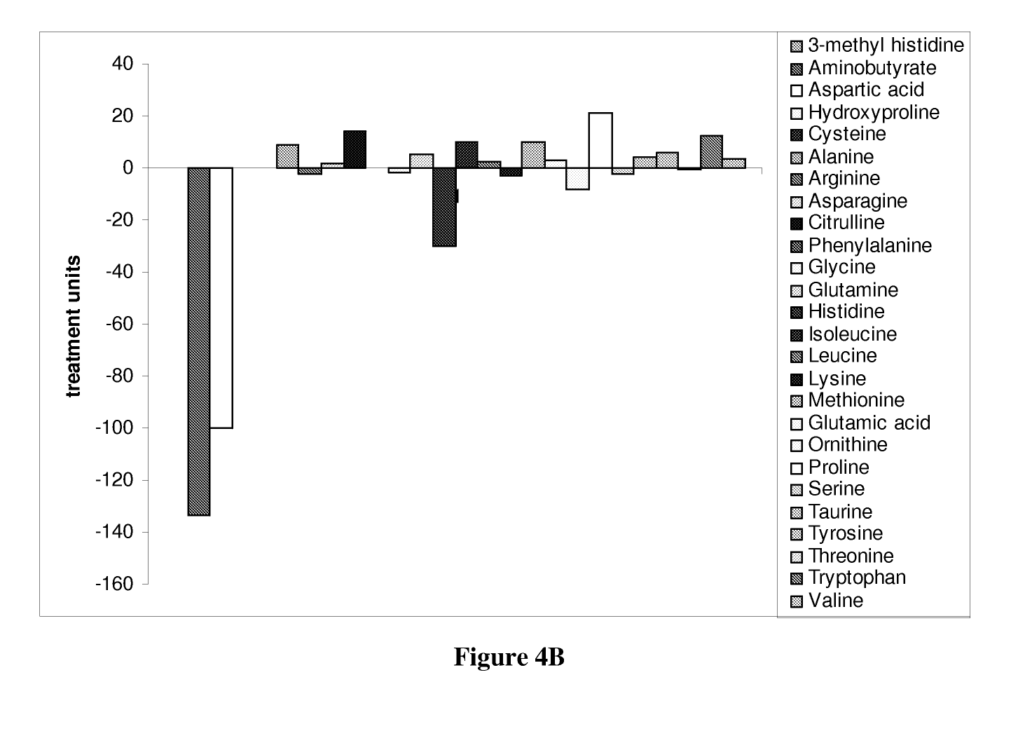

Reference is now made to a dietary composition formulated according to an AA profile of a patient's tumor. FIG. 4A is a plotted-graph depiction of an exemplary amino-acid profile of a tumor sample, according to preferred embodiments of the present invention. An exemplary AA profile for patients with a lung tumor disease is shown. A profile of 26 amino acids is presented. The measured AA values have been normalized according to the corresponding AA values of a healthy, control group. Therefore, the presented profile shows the difference in the AA value ratio of tumor patients over healthy patients. According to certain embodiments of the present invention, such a profile provides the AA specificity of an individual tumor type.

FIG. 4B is a plotted-graph depiction of an exemplary computer-generated profile for a dietary composition formulated according to the amino-acid profile of FIG. 4A, according to preferred embodiments of the present invention. The profile is used to formulate a dietary composition specific to the tumor type.

It is within the scope of the present invention that the treatment profile is in inverse correlation to the AA profile or pattern of the specific tumor, or in direct correlation modified by a constant. For example, as shown in FIG. 4A, the amino acid 3-methyl histidine shows the highest positive difference relative to the AA level of the healthy group (“normal”). In the treatment profile shown in FIG. 4B, the level of 3-methyl histidine is the lowest. On the other hand, the amino acid proline shows the highest negative difference from normal (in FIG. 4A); therefore, its level in the treatment profile (in FIG. 4B) is the most significantly elevated.

In accordance with embodiments of the present invention, the treatment units of an amino acid or other profiled biological molecule are determined by an algorithm or processing system. Such an algorithm or processing system determines the treatment unit by calculating the ratio between the difference in the measured AA value (or other, profiled biological-molecule value) of a healthy individual and a tumor patient, and the measured AA value (or other, profiled biological-molecule value) of a healthy individual.

In accordance with further embodiments of the present invention, the treatment profile of an individual tumor is determined by processing the biochemical profile data of a tumorous tissue in comparison to the corresponding, profile data of healthy tissue of the same individual.

It is herein emphasized that tumors of different organs may differ, not only in their capacity for proliferation and metastasis, but also in their metabolic status and consequently in their biochemical profile (e.g. in their AA profile). As shown in FIGS. 4A-B, the biochemical profiling of a specific tumor enabled a dietary composition and a treatment regime specific to retard the development of a specific tumor to be tailored.

### Example 4

In preferred embodiments of the present invention, AA formulations for breast cancer, prostate cancer, lung cancer, and colon cancer are provided on the basis of cancer types. Refined prescriptions are provided according to personalized tests.

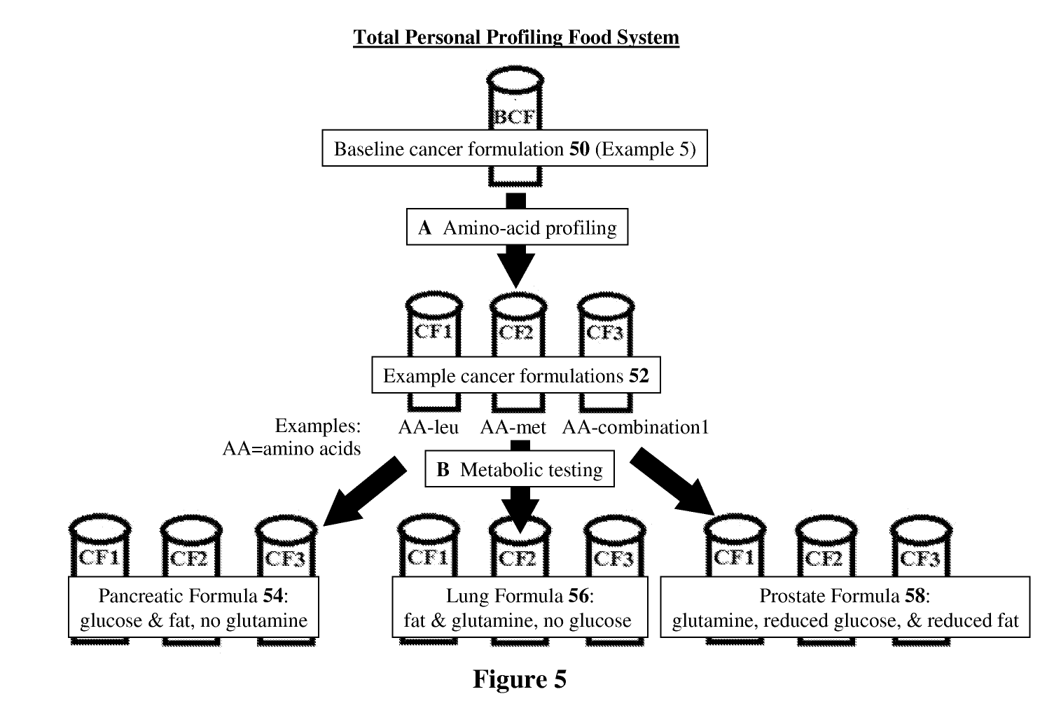

Breast cancer: depleted or reduced AA concentration of at least 50% reduction from normal consumption to depletion (see Table 1 below) of one or more of the following: Arginine (Arg), Glutamine (GLN), and Methionine (Met) (for Stage I), and Met, Asparagine (Asn), Phenyl-alanine (Phe), and Histidine (His) (for Stage III). Glucose should be depleted from the food (see FIG. 5), and fat consumption should be limited to omega-3 and coconut oil (25%).

Prostate cancer: depleted or reduced AA concentration of at least 50% reduction from normal consumption to depletion (see Table 1 below) of one or more of the following: GLN, Glycine (GLY), Tryptophan (TRP), Arg (for Glison level 6-7) and Leu, His, Trp, Met, and Arg (Glison level 8-10). Glucose should be restricted (see FIG. 5), and fat consumption should be limited to omega-3, and coconut oil (25%).

Lung cancer (small cell and non-small cell): depleted or reduced AA concentration of at least 50% reduction from normal consumption to depletion (see Table 1) of one or more of the following: His, GLN, Asn, and Cys (for Stage I), and Leu, Met, Cys, Gln, Asn, His, and Trp (for Stage IV). Glucose and carbohydrates should be depleted.

Colorectal cancer: depleted or reduced AA concentration of at least 50% reduction from normal consumption to depletion (see Table 1 below) of one or more of the following: Threonine (Thr), Gly, Met, Cys, Phe, Tyr, and Trp (for Stage I), and Trp, Asn, Val, and Met (for Stage IV). Fats and carbohydrates should be prescribed based on a individual, metabolic test results. Glutamine can be added in excess.

### Example 5

### Baseline Formulation

A baseline formulation is the basic platform on which the rest of the formulations are developed. The baseline formulation contains basic minerals, nutrients, and functional foods that are known to be beneficial drug supplements for cancer patients. A non-limiting example is the following (values in milligrams, unless stated otherwise): liloneic acid 3500, lipoic acid 600, 1-carnitine 900, vitamin D3 2000, vitamin K2 1000, calcium 500, vitamin A 400, thiamin 1900, vitamin B2 900, vitamin B6 750, vitamin B12 20 mcg, niacin 10000 mcg, folic acid 230, pantothenic acid 6900, biotin 65, choline 80, magnesium 50, iron 9, zinc 10, selenium 20, manganese 500 mcg, copper 100 mcg, iodine 65, omega 3 as “mila hispanica L” 10 gr, green tea extract 500, turmeric 1000, molybdenum 12, sodium 190, potassium 300, chloride 323, phospate 400, and inositol 40. Optionally, arctigenin and reservatrol can be added to the formulation as well.

There are three variations to the baseline formula with varied amounts of amino acids:

1. Fat-rich—fat content should be 21-50 gr. and contain sunflower oil, coconut oil, and flaxseed oil (non-oxidized);

2. Glucose-rich—glucose source from fruits rich in glucose (no glutamine); and

3. Glutamine-rich.

The basic AA structure is provided according to the regulations by WHO from 1985, shown in Table 1 below, or following the formula described in U.S. Pat. No. 5,242,697.

TABLE 1
Standard concentrations of essential amino acids
in food consumption per day

| | 1985 FAO/WHO |
|---|---|
| Essential amino acid | Requirement |
| Isoleucine | 10 (0.12) |
| Leucine | 14 (0.17) |
| Lysine | 12 (0.14) |
| Methionine & Cystine | 13 (0.16) |
| Phenylalanine & Tyrosine | 14 (0.17) |
| Threonine | 7 (0.08) |
| Tryptophan | 3.5 (0.04) |
| Valine | 10 (0.12) |
| TOTAL | 83.5 (1.00) |

*AA requirement for adults in mg/kg/day (number in brackets is a relative fraction of the amino acid in the total)

### Example 6

### AA Deprivation-Formulation Profiles for Head and Neck Cancer Patients

M. Cobo et al., Oncologia, 2006, 29 (7) 283-290, have found that there are significant alterations in the AA profiles of lung, head, and neck patients relative to a healthy control group. Baseline serum levels of 27 amino acids were analyzed in 51 patients with cancer of the lung or head/neck with no metabolic alterations or other concomitant disorders, and compared with the results of a control group. It was found that. compared with the control group, patients with head cancer had significant differences in cysteine, aspartic acid, 3-methyl histidine, alanine, glycine, lysine, methionine, proline, serine, taurine, tyrosine, and threonine; while patients with lung cancer had significant differences in cysteine, aspartic acid, 3-methyl histidine, histidine, citrulline, ornithine, alanine, glycine, lysine, methionine, proline, serine, taurine, tyrosine, and threonine. The exact values are provided in Table 2.

Based on the data in Table 2, a basic dietary formula for head and neck cancer patients that contains basic nutrients, vitamins, and minerals as described in Example 5 in addition to the AA composition is follows: all essential L-amino acids and non-essential amino acids in standard amounts according to the WHO (1985 regulations), except for one or more of the following with altered concentrations: cysteine and methionine reduced to about 50% or less until total depletion according to the patient's personal profile, L-leucine reduced to about 50% or less until total depletion according to the patient's personal profile, aspartic acid reduced to about 50% or less until total depletion according to the patient's personal profile, and tyrosine reduced to about 50% or less until total depletion according to the patient's personal profile. Optionally, arctigenin can be added to the formulation as well.

### Example 7

Maeda et al., BMC Cancer, 2010; 10: 690, have studied plasma-free AA concentrations of non-small-cell, carcinoma patients relative to a control group. The AA balance in cancer patients was observed to often differ from that in healthy individuals because of metabolic changes. The study investigated the use of plasma AA profiles as a novel marker for screening non-small-cell, lung cancer (NSCLC) patients. In this example, the results of the screening study are used to construct an AA deprivation scheme for the patient that correlate with the AA plasma concentrations.

The results of the study show that His levels were significantly lower than the control, and the levels of serine, praline, glycine, alanine, methionine, isolucine, leucine, tyrosine, phenylalanine, ornitine, and lysine were significantly higher than the control. The results further show that the amino acids that vary in concentrations are sensitive to stress, and release heat shock proteins, or undergo autophagy, to maintain high amounts of the amino acids that the tumor needs. Therefore, the depleted AA formula basically contains histidine in reduced (50% or less) or depleted amounts, leucine- and methionine-free combinations to interfere with autophagy, and optionally, arctigenin to interfere with the starving cells obtaining food from their environment.

### Example 8

### Total Personal Profiling Food System

FIG. 5 is a high-level schematic block diagram of a total personal profiling food system, showing processing steps, for creating personal combinations for patients according to their cancer type and profile, according to preferred embodiments of the present invention. A Baseline cancer formulation 50 (BCF), such as the formulation described in Example 5, is constant and can be provided as soup, soup mix, powder, or liquid. Baseline cancer formulation 50 is augmented with cancer formulations 52 (CF1-3) which are based on AA profiling (Process Step A).

Cancer formulations 52 contain personalized, reduced or depleted combinations of amino acids such as: Methionine, Leucine, Arginine, Cysteine, Asparagine, Histidine, and Glutamine. Examples of cancer formulations 52 such as depleted amino acids Leu, Met, and a combination are shown in FIG. 5.

Based on metabolic testing (Process Step B), cancer type-specific formulations can be determined such as: pancreatic formula 54 having glucose and fat with no glutamine, lung formula 56 having fat and glutamine with no glucose, and prostate formula 58 having glutamine, reduced glucose, and reduced fat. The metabolic tests and tumor type provide indications of how the tumor produces its energy: (1) through aerobic glycolysis, (2) through beta-oxidation, (3) through glutamine, and (4) through a combination of (1) and (2). In case (1), glucose should be deprived. In case (2), fats should not be consumed. In case (3), glutamine should not be consumed. In case (4), glutamine should be used as a source of energy. In all cases, arctigenin may be added to the treatment to lower the tolerance of the cancer cells to starvation, and cause cell death.

### Example 9

FIG. 6 is a simplified flowchart of the major operational steps for optimizing and monitoring the treatment, changing the formulation with progression of treatment, and formulating and administering the diet, according to preferred embodiments of the present invention. The process involves administering a diet regime or composition to a patient in a therapeutically-effective dosage (Step 60). The amino acids of cancerous tissue are profiled using an MS or AA analyzer (Step 62), and the tumor is then monitored for the indication of a deprivation state using a bioanalytical tool (e.g. blood and plasma samples via MRI) (Step 64).

A deprivation state is detected according to the presence of ATG4 initiator of autophagy, HSP70, or AKT1 (e.g. free nitrogen test to monitor starvation/autophagy detection) (Step 66). A second-stage food regime is provided containing a deprivation formula, botanicals such as arctigenin or other AKT1 inhibitors, and cytotoxic materials such as PES (Phenylethynesulfonamide) (Step 68). A diet regime or composition is then administered to the patient in a therapeutically-effective dosage as a meal replacement or supplement to a diet (Step 70), followed by long-term maintenance.

### Example 10

### Diagnostic Tests

Diagnostics tests are performed as part of the process to determine the personalized formula of combined AA deprivation and metabolic deprivation for cancer patients in order to reduce tumor size. The different diagnostic tests performed are listed include: AA composition of tumor tissue; AA composition of blood plasma; metabolic pathway using metabolomics and metabolites; specific markers for monitoring starvation, HSP, AKT1, or autophagy, or consumption in blood; urine nitrogen; monitoring different markers for tracking starvation and cell death (e.g. biotin, trehalose, ergothionine, S-adenosylmethionine, CDP choline, creatinine, glutamine, sodium selenite, silver water, TorB, BRCA1, and PSA).

### Example 11

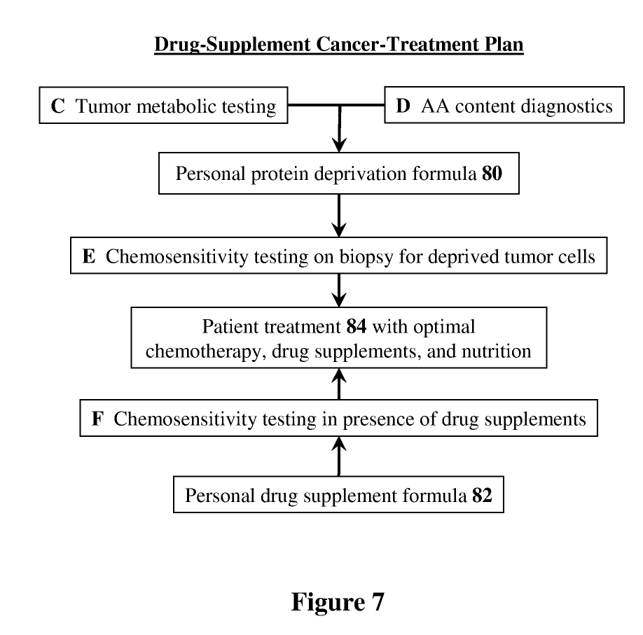

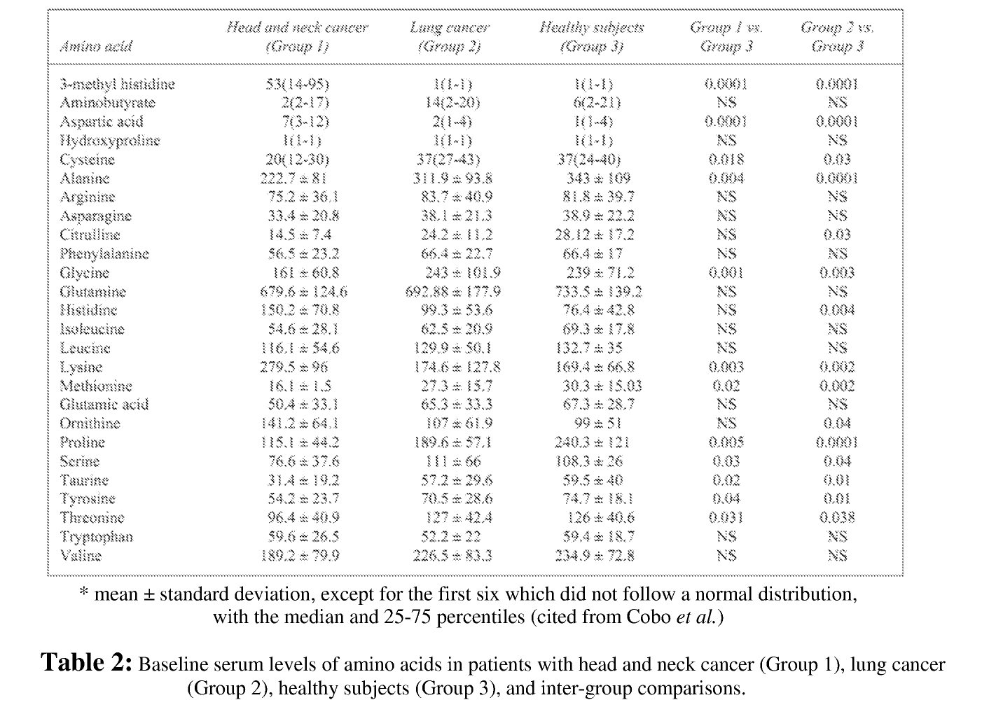

FIG. 7 is a simplified flowchart of the major operational steps for a drug-supplement cancer-treatment plan using an AA starvation diet and drug supplements in-vitro to derive an optimal chemotherapy, drug supplement, diet plan, according to preferred embodiments of the present invention. Tumor metabolic testing (Process Step C) and AA content diagnostics (Process Step D) are performed to prescribe a personal protein deprivation formula 80. Chemosensitivity testing on biopsy for monitoring deprived tumor cells is then performed (Process Step E).

In conjunction with this protocol, a personal drug supplement formula 82 is provided to the cancer patient. Chemosensitivity testing is affected by the presence of drug supplements (Process Step F). From such a holistic approach, a patient treatment 84 is obtained having optimal chemotherapy, drug supplements, and nutrition components.

While the present invention has been described with respect to a limited number of embodiments, it will be appreciated that many variations, modifications, and other applications of the present invention may be made.

What is claimed is:

1. A method for determining a diet regime for a patient with a tumor disease, the method comprising the steps of:
(a) providing a sample of the patient;
(b) profiling at least one biochemical parameter of said sample using a biochemical analyzer to obtain a profile;
(c) identifying a biologically-active molecular feature of said profile;
(d) correlating said feature with a biochemical pathway related to the tumor's metabolism or proliferation;
(e) determining the diet regime specific to the patient, wherein the diet regime includes at least one biologically-active molecule corresponding to said feature of said profile; and
(f) administering the diet regime to the patient in a therapeutically-effective dosage.

2. The method of claim 1, wherein said sample is selected from the group consisting of: a tumor sample, biological tissue, an organ sample, blood, blood serum, blood plasma, and urine.

3. A method for diagnosing a tumor in a subject, the method comprising the steps of:
(a) providing a sample of the subject;
(b) profiling at least one biochemical parameter or at least one biophysical parameter of said sample using a biochemical analyzer or a biophysical analyzer to obtain a profile;
(c) storing results of said profile in a profile database; and
(d) processing said results by comparing said results with profile data in said database, thereby diagnosing the tumor.

4. The method of claim 3, wherein said sample is selected from the group consisting of: a tumor sample, biological tissue, an organ sample, blood, blood serum, blood plasma, and urine.

5. The method of claim 3, wherein said step of processing includes calculating the ratio between a difference in a profiled, biological-molecule value of a healthy individual and a tumor patient, and a healthy biological-molecule value of a healthy individual.

6. The method of claim 3, wherein said step of processing includes comparing biochemical profile data of said sample with corresponding profile data of healthy tissue of the subject.

7. The method of claim 3, wherein said profile is selected from the group consisting of: a chemosensitivity profile, an amino-acid profile, a genomic profile, a deprivation-state profile, a genetic-marker profile, a gene-expression profile, a transcriptome profile, a proteomics profile, a metabolomics profile, a pharmacogenomics profile, pharmacokinetics profile, an electromagnetic-frequency profile, an electrochemical profile, a fatty-acid profile, a carbohydrate profile, a lipid profile, an oligosaccharide profile, a metabolite profile, a chemical profile, an organic-ion profile, or an inorganic-ion profile, a free-radical profile, a bioimpedance profile, a conductivity profile, a voice-analysis profile, a biomarker profile, and any combination thereof.

8. A dietary composition for a patient with a tumor, the composition comprising depleted or reduced amino-acid concentrations of at least 50% reduction from normal consumption to depletion of at least one amino acid selected from the group consisting of: Arginine (Arg), Glutamine (Gln), Methionine (Met), Asparagine (Asn), Phenylalanine (Phe), Histidine (His), Glycine (Glt), Tryptophan (Trp), Leucine (Leu), Threonine (Thr), Valine (Val), Cystine (Cys), Isoleucine (Iso), Lysine (Lys), Aspartic acid (Asp), and Tyrosine (Tyr).

9. The dietary composition of claim 8, wherein said tumor is associated with breast cancer, and wherein the dietary composition comprises depleted or reduced amino-acid concentrations of at least 50% reduction from normal consumption to depletion of at least one of: Arg, Gln, Met, Asn, Phe, and His.

10. The dietary composition of claim 8, wherein said tumor is associated with prostate cancer, and wherein the dietary composition comprises depleted or reduced amino-acid concentrations of at least 50% reduction from normal consumption to depletion of at least one of: Gln, Gly, Trp, Arg, Leu, His, and Met.

11. The dietary composition of claim 8, wherein said tumor is associated with lung cancer, and wherein the dietary composition comprises depleted or reduced amino-acid concentrations of at least 50% reduction from normal consumption to depletion of at least one of: His, Gln, Asn, Cys, Leu, Met, and Trp.

12. The dietary composition of claim 8, wherein said tumor is associated with colorectal cancer, and wherein the dietary composition comprises depleted or reduced amino-acid concentrations of at least 50% reduction from normal consumption to depletion of at least one of: Thr, Gly, Met, Cys, Phe, Tyr, Trp, Asn, and Val.

13. The dietary composition of claim 8, wherein said tumor is associated with head and neck cancer, and wherein the dietary composition comprises depleted or reduced amino-acid concentrations of at least 50% reduction from normal consumption to depletion of at least one of: Met, Cys, Tyr, Leu, and Asp.

14. The dietary composition of claim 8, wherein said at least one molecule is selected from the group consisting of: a precursor of said at least one feature, an antagonist of said at least one feature, an inhibitor of said at least one feature, a participant in a biochemical pathway associated with said at least one feature, a cofactor of said feature, a participant in a biochemical pathway associated with said metabolism or said proliferation, and a biomarker of a tumor disease.

15. The dietary composition of claim 8, wherein said biochemical pathway is selected from the group consisting of: of altered gene expression, gene mutagenesis, metastasis, apoptosis, programmed cell death, autophagy angiogenesis, growth factor regulation, receptor regulation, signal transduction, cell proliferation, cell migration, cell adhesion, cell expansion, cell differentiation, cell invasion, tissue progenitors regulation, cell death, aging process, cellular senescence, carcinogenesis, DNA repair, DNA-damage responses, tumorigenesis, anaplasia, abnormal protein synthesis and expression, genomic instability, neoplasia, thrombosis, hyperplasia, dysplasia, aneuploidy, genomic amplification, variation in nuclear size and shape, abnormal tissue organization, growth signals, immortality, mitosis, cell cycle regulation, homeostasis, transcription, haploinsufficiency, telomerase mutations, telomerase mutations, oxidative stress, hypoxia, hyperprolactinemia DNA methylation, pleomorphism, atypia, necrosis, meningitis, astrocytoma, glioblastoma multiforme, deprivation state target, autophagy and any combination thereof.

16. A dietary composition for a patient with a tumor, the composition comprising: at least one biologically-active molecule corresponding to at least one biologically-active molecular feature of a biochemical tumor profile, wherein said at least one feature correlates with at least one biochemical pathway related to metabolism or proliferation of the tumor.

17. The dietary composition of claim 16, wherein said at least one feature is either reduced, lacking, or in excess in said tumor profile relative to a healthy profile of corresponding healthy tissue.

18. The dietary composition of claim 16, wherein an identity and/or dosage of said at least one molecule is either in direct correlation or in inverse correlation with said at least one feature or in correlation modified by a constant thereof.

19. The dietary composition of claim 16, wherein said at least one molecule is selected from the group consisting of: a precursor of said at least one feature, an antagonist of said at least one feature, an inhibitor of said at least one feature, a participant in a biochemical pathway associated with said at least one feature, a cofactor of said feature, a participant in a biochemical pathway associated with said metabolism or said proliferation, and a biomarker of a tumor disease.

20. The dietary composition of claim 16, wherein said molecule is adapted to cause cells in said tumor to starve, resulting in signals and/or processes to be triggered, and wherein said signals and/or processes include AKT, HSP, HSP70, autophagy, and apoptosis.

21. The dietary composition of claim 16, wherein said biochemical pathway is selected from the group consisting of: of altered gene expression, gene mutagenesis, metastasis, apoptosis, programmed cell death, autophagy angiogenesis, growth factor regulation, receptor regulation, signal transduction, cell proliferation, cell migration, cell adhesion, cell expansion, cell differentiation, cell invasion, tissue progenitors regulation, cell death, aging process, cellular senescence, carcinogenesis, DNA repair, DNA-damage responses, tumorigenesis, anaplasia, abnormal protein synthesis and expression, genomic instability, neoplasia, thrombosis, hyperplasia, dysplasia, aneuploidy, genomic amplification, variation in nuclear size and shape, abnormal tissue organization, growth signals, immortality, mitosis, cell cycle regulation, homeostasis, transcription, haploinsufficiency, telomerase mutations, telomerase mutations, oxidative stress, hypoxia, hyperprolactinemia DNA methylation, pleomorphism, atypia, necrosis, meningitis, astrocytoma, glioblastoma multiforme, deprivation state target, autophagy and any combination thereof.

22. The dietary composition of claim 21, wherein said altered gene expression or mutagenesis is associated with genes selected from the group consisting of: oncogenes, tumor suppressor genes, growth factor genes, angiogenic factor genes, receptor genes, and any combination thereof.

23. The dietary composition of claim 16, wherein said at least one molecule is selected from the group consisting of: amino acids, precursors of amino acids, antioxidants, fatty acids, lipids, proteases, protease inhibitors, antagonists, carbohydrates, oligosaccharides, vitamins, nutrients, ions, minerals, trace elements, cofactors, enzymes, enzyme inhibitors, and a mixture thereof.

24. The dietary composition of claim 16, the composition further comprising: a predetermined amino-acid content correlated with the amino-acid profile of said tumor.

25. The dietary composition of claim 24, the composition further comprising: at least one composition attribute selected from the group consisting of: complex carbohydrates, 0% fat, 0% glucose, 0% fructose, and 0% carbohydrates, arctigenin, at least one depleted amino acid, at least one depleted amino-acid precursor, at least one depleted metabolite involved in synthesis of said amino acid.

26. The dietary composition of claim 16, the composition further comprising: at least one depleted, essential amino acid selected from the group consisting of: Isoleucine, Leucine, Lysine, Methionine, Phenylalanine, Threonine, Tryptophan and Valine, a precursor thereof, a metabolite involved in synthesis of said amino acid, and enzymes capable of decomposing said amino acid.

27. The dietary composition of claim 16, the composition further comprising: at least one depleted, non-essential amino acid selected from the consisting of: Alanine, Asparagine, Aspartic acid, Cysteine, Glutamic Acid, Glutamine, Glycine, Proline, Selenocysteine, Serine, Tyrosine, Arginine, Histidine, Ornithine, and Taurine, a precursor thereof, and a metabolite involved in synthesis of said amino acid.

28. The dietary composition of claim 16, wherein said tumor profile is associated with a tumor selected from the group consisting of: a sarcoma, carcinoma, lymphoma, myeloma leukemia, and cancers of the central nervous system (CNS).

29. The dietary composition of claim 16, the composition adapted to provide a synergistic therapeutic effect with respect to inhibition of tumor metabolism and/or proliferation when administered in combination with a conventional anti-tumor drug or treatment to said patient.

30. A method for optimizing and administering a diet treatment for a patient with a tumor disease, the method comprising the steps of:
(a) administering a dietary composition to the patient in a therapeutically-effective dosage;
(b) profiling amino acids of a tumor of the patient using a biochemical analyzer;
(c) monitoring said tumor for indication of a deprivation state using a bioanalytical tool;
(d) detecting said deprivation state of said tumor; and
(e) second-stage administering of a modified deprivation diet in a therapeutically-effective dosage, wherein said modified deprivation diet includes said dietary composition, at least one participant in a biochemical pathway associated with metabolism or proliferation of said tumor, and at least one cytotoxic material.

31. A system for processing tumor-related profile data, the system comprising:
(a) a CPU for performing computational operations;
(b) a memory module for storing data;
(c) a profile-analysis module for profiling at least one biochemical parameter or at least one biophysical parameter of a tumor sample;
(d) a profile-processing module for processing profiling results of said at least one biochemical parameter or said at least one biophysical parameter; and
(e) a protocol-generating module for generating a protocol, for a dietary composition based on said processing of said profiling results.

32. A non-transitory computer-readable medium, having computer-readable code embodied on the non-transitory computer-readable medium, the computer-readable code comprising:
(a) program code for receiving a profile of at least one biochemical parameter or at least one biophysical parameter of a tumor sample of a subject using a biochemical analyzer or a biophysical analyzer;
(b) program code for storing results of said profile in a profile database; and
(c) program code for processing said results by comparing said results with profile data in said database, thereby diagnosing said sample.
